(19) United States
(12) Patent Application Publication
(10) Pub. No.: US 2018/0311201 A1
(43) Pub. Date: Nov. 1, 2018

(54) ORAL PHARMACEUTICAL COMPOSITION COMPRISING OTILONIUM BROMIDE AND SIMETHICONE WITH CERTAIN BULK DENSITY AND IMPROVED DISSOLUTION CHARACTERISTICS

(71) Applicant: SANTA FARMA ÍLAÇ SANAYÍ, Sisli/Istanbul (TR)

(72) Inventors: Ahmet GÛN, Istanbul (TR); Banu Öztuna, Istanbul (TR); Bayram Kanik, Istanbul (TR); Ediz AKZOY, Istanbul (TR); Levent ÜNVER, Istanbul (TR); Levent ÖNER, Ankara (TR)

(73) Assignee: SANTA FARMA ?LAÇ SANAY? A. ?., ?stanbul (TR)

(21) Appl. No.: 15/735,607

(22) PCT Filed: Jun. 12, 2015
(86) PCT No.: PCT/TR2015/000259

§ 371 Date: Dec. 12, 2017

(51) Int. Cl. A61K 31/245; A61K 31/695; A61K 9/20

(52) U.S. Cl. CPC A61K 31/245; A61K 31/695; A61K 9/2054; A61K 9/2027; A61K 9/2059

(57) ABSTRACT

The present invention relates to a pharmaceutical composition for oral use comprising therapeutically effective amount of otilonium bromide in combination with therapeutically effective amount of simethicone, wherein the composition possesses certain bulk density and improved dissolution characteristics. Wherein a final blend prior to compression has a bulk density of at least 0.35 g/mL, and at least 85% of the otilonium bromide is released in 15 minutes in each of 0.1 N HCl, pH 4.5 and pH 6.8 buffer solutions at 37±0.5° C. using USP type II (paddle) apparatus rotating at 50 rpm.

### CROSS REFERENCE TO RELATED APPLICATIONS

This application is the national phase entry of International Application No. PCT/TR2015/000259, filed on Jun. 12, 2015, the entire contents of which are incorporated herein by reference.

### TECHNICAL FIELD

The present invention relates to a pharmaceutical composition for oral use comprising therapeutically effective amount of otilonium bromide in combination with therapeutically effective amount of simethicone or pharmaceutically acceptable forms or derivatives thereof, wherein simethicone is used either in powder or in liquid form. More specifically, the present invention relates to a solid oral dosage form comprising therapeutically effective amount of otilonium bromide and simethicone in an acceptable carrier, wherein the invented composition possesses certain bulk density and improved dissolution characteristics.

The present invention further relates to solid dosage forms for oral administration such as tablet, film-coated tablet, dispersible tablet, orally disintegrating tablet, powder, granules or a combination thereof. This invention also relates to method of making the aforesaid solid oral dosage forms.

### BACKGROUND

The present invention relates to a pharmaceutical composition for oral use comprising therapeutically effective amount of otilonium bromide in combination with therapeutically effective amount of simethicone, wherein simethicone is used either in powder or in liquid form, and wherein the invented formulation possesses certain bulk density and improved dissolution characteristics. Such combination can be used to treat patients suffering from irritable bowel syndrome (MS) and/or pain and spasm of the distal enteric tract and/or painful symptoms of excessive gas in gastrointestinal (GI) tract.

Otilonium bromide (Formula I), N,N-Diethyl-N-methyl-2-(4-[2-(octyloxy)benzamido]benzoyl oxy)ethanaminium bromide, is used in symptomatic treatment of irritable bowel, pain and spasm of the distal enteric tract. Not being reported in any pharmacopoeia, the empirical formula of otilonium bromide is C₂₉H₄₃BrN₂O₄. It is reported to be soluble in water (12 mg/mL), in dimethyl sulfoxide (>25 mg/mL) and in ethanol.

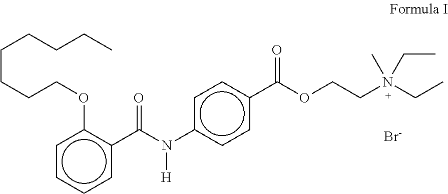

Otilonium bromide is known for its marked spasmolytic action on the smooth muscle of the digestive tract, and it has good tolerability profile which acts by modifying Ca′ fluxes from extra- and intra-cellular sites. Following oral administration otilonium bromide has poor systemic absorption and exerts its activity principally on distal GI tract. It accumulates in the lower intestine and its spasmolytic effect has a direct action on the contractile proteins of the smooth muscle. The recommended dose of otilonium bromide is 80-120 mg daily by the oral administration of a single tablet containing 40 mg of otilonium bromide 2-3 times a day.

The original product containing 40 mg of otilonium bromide as the active substance is commercially available by Menarini under various trade names, e.g., Spasmomen® in Italy, Portugal, Belgium, Czech Republic, Hungary, Turkey; Doralin® in Greece; Spasmoctyl® in Spain. Spasmomen®/Spasmoctyl® tablets contain lactose, starch, sodium starch glycolate, magnesium stearate, hydroxypropylmethyl cellulose, titanium dioxide and polyethylene glycol and are prepared by granulation techniques.

WO 2011/024028 A1 (Abdi Ibrahim) relates to direct compression of otilonium or its pharmaceutically acceptable salt.

EP 2481395 A1 (Deva Holding) discloses sachet, effervescent, dry syrup formulations of otilonium or pharmaceutically acceptable salts and as well as processes of preparation of these dosage forms.

CN 101053562 A (Beijing Dezhong Wanquan) relates to pharmaceutical compositions comprising 5-100 mg of otilonium bromide, where the preferred pharmaceutical dosage form is capsule and said composition disintegrates within five minutes in water.

Simethicone (Formula II) is a mixture of fully methylated linear siloxane polymers containing repeating units of dimethylpolysiloxane stabilized with trimethylsiloxy end-blocking units, and silicon dioxide, which contains 90.5-99% of dimethylpolysiloxanes (DMPS) and 4-7% of silicon dioxide. The DMPSs present in simethicone are essentially inert polymers having a molecular weight of 14,000-21,000. Its empirical formula is (C₂H₆OSi)ₙ.(SiO₂)ₓ, where n (the degree of polymerization) varies from 20 to 400.

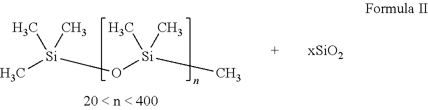

Simethicone is globally known for its use in pharmaceutical formulations not only as an excipient but also as an active ingredient. It is a surface active agent which acts as a defoamer or dispersent of gas bubbles by changing the surface tension of the bubbles to enable them to coalesce. Simethicone acts largely in the stomach but is also believed to have gas relieving effect in the intestines. Its defoaming action relieves flatulence by dispersing and preventing the formation of mucous surrounded gas pockets in the GI tract. Simethicone does not decrease the amount of gas in the digestive tract, but by decreasing the size and surface tension of gas bubbles it increases the rate at which gas leaves the body, hence relieves the pain and pressure commonly associated with the presence of excessive gas in the GI tract. Since it is not absorbed or metabolized by the body following oral administration, simethicone is considered relatively safe throughout the GI tract.

The preferred dose of simethicone is in the range of about 20 to 125 mg per dosage unit, generally not to exceed 500 mg/day. The dose ranges may vary for age and weight of a patient as well as the severity of symptoms.

EP 0891776 B1 (McNeil-PPC, Inc.) discloses oral solid dosage form preparations formed from a free flowing granular composition comprising simethicone and granular anhydrous tribasic calcium phosphate and/or dibasic calcium phosphate which is suitable for compression into a solid dosage form for oral administration.

EP 0425450 B1 (Valentine Enterprises Inc.) discloses simethicone preparations that are in the form of combinations of water soluble agglomerated maltodextrin and simethicone.

WO 01/41737 A2 (Shire Laboratories, Inc.) discloses an solid oral dosage form wherein a single solid carrier such as magnesium aluminosilicate, or granulated dibasic calcium phosphate is used to absorb simethicone.

EP1297825 B1 (McNeil-PPC, Inc.) discloses admixture compositions of simethicone, an adsorbent, and an optional active agent in which the weight ratio of the simethicone to adsorbent is 1:1.75 to 1:2.22.

Simethicone has been utilized in a variety of therapeutic liquid and solid dosage forms. Examples of commercial products containing simethicone are Gas-X® Regular/Extra/Ultra Strength chewable tablets by Novartis, Dulcogas™ Sachet by Boehringer Ingelheim, and Sab Simplex® Suspension by Pfizer and Dentinox Infant Colic Drops by DDD Limited.

Simethicone has also been investigated in a variety of combinations with other active ingredients.

EP 00142 53 B1 (Joseph A. Rider) relates to a tablet containing at least two separate layers one of which contains 10-100 mg of simethicone and the other of which contains 100-1000 mg of antacid.

EP 0428296 B1 (McNeil-PPC, Inc.) relates to a pharmaceutical composition for treating GI distress comprising loperamide in a dosage range of about 0.5-8.0 mg and simethicone in a dosage range of about 20-125 mg.

EP 0571217 B1 (McNeil-PPC, Inc.) relates to multilayered oral dosage forms comprising loperamide and simethicone.

WO 2008/056200 A1 (Ranbaxy Lab Ltd.) relates to oral pharmaceutical compositions comprising simethicone, a pharmaceutically acceptable salt of silicate (e.g., calcium silicate), at least one adsorbent material that adsorbs simethicone, and optionally one or more pharmaceutically acceptable excipients.

WO 2013/095111 A1 (Disphar International BV) discloses a pharmaceutical composition comprising a mixture of simethicone, calcium phosphate powder and mannitol, wherein calcium phosphate powder and mannitol act as carriers for simethicone liquid to form a solid-liquid blend. The said composition may further comprise a second active ingredient, such as loperamide.

Commercially, simethicone is available in combination with other active ingredients, such as loperamide (i.e., Imodium® Plus Caplets, Imodium® Plus Chewable Tablets and Imodium® Plus Comfort Tablets by McNeil Products Ltd.; Losikole 2 mg/125 mg tablets by Disphar International B.V.) and hydrotalcite (i.e., Altacite Plus® Suspension by Peckforton Pharmaceuticals).

Similarly, otilonium bromide is commercially available in combination with other active ingredients, such as diazepam (i.e., Spasmomen Somatico® by Menarini).

EP 2481403 A1 (Deva Holding) discloses pharmaceutical combinations of otilonium bromide and trimebutine maleate.

EP 0270503 B1 (Menarini) discloses a new pharmaceutical form of otilonium bromide which is suitable to be applied locally in the GI tract, i.e., a bottle containing otilonium bromide as the active substance together with one or more antifoaming excipients (e.g., dimethylpolysiloxane=DMPS=dimeticone) and one or more emulsifying excipients and a solvent vial containing sterile water. Yet this invention relates to pharmaceutical composition suitable for topical application rather than oral administration, where dimeticone rather than simethicone is used, and moreover it is used as an inactive substance (i.e., as antifoaming agent) rather than an active substance.

WO 2010/092436 A1 (Abdi Ibrahim) relates to pharmaceutical combinations of simethicone typically presented in an amount from about 10 mg to about 240 mg and otilonium bromide typically presented in an amount from about 10 mg to about 150 mg, where simethicone is used preferably in amount of 80 mg and otilonium bromide is used preferably in amount of 40 mg. Yet this invention does not address the dissolution properties of the combination product. Moreover, no information with regard to compressibility or bulk/tapped density is disclosed in this document.

TR 2013/00014 and TR 2013/017017 (Abdi Ibrahim) relate to pharmaceutical combinations of simethicone and otilonium bromide, where simethicone is used preferably in amount of 80 mg and otilonium bromide is used preferably in amount of 40 mg, where each active ingredient is granulated in separate portions and then compressed into bilayer tablets comprising various inactive substances. However, none of these inventions refer to or disclose dissolution properties of the said bilayer tablets comprising otilonium bromide and simethicone as active substances. Moreover, no information with regard to compressibility or bulk/tapped density is disclosed in these documents.

TR 2014/01826 (Santa Farma) discloses encapsulated pharmaceutical combinations of otilonium bromide and simethicone, where each capsule contains otilonium bromide in tablet form and simethicone in granular form, and preferably the amount of otilonium bromide and simethicone are 40 mg each. This invention does not refer to or disclose dissolution properties of the said encapsulated compositions.

The combination of otilonium bromide and simethicone has been registered in Turkey by Abdi Ibrahim (Trade name: Ekspaz Plus Film Tablet) containing 40 mg of otilonium bromide and 80 mg of simethicone. The excipients listed in the Summary of Product Characteristics (SmPC) of Ekspaz Plus Film Tablet are silicified microcrystalline cellulose (Prosolv® SMCC 90), lactose granule, colloidal silicon dioxide (Aerosil® 300 & Aerosil® 200), magnesium stearate, copovidone (Kollidon® VA 64 Fine), crospovidone (Kollidon® CL), and Opadry® AMB Yellow 80W22002 (mixture of lecithin, titanium dioxide, xanthan gum, talc, yellow iron oxide, polyvinyl alcohol). Ekspaz Plus Film Tablet (Abdi Ibrahim) has not been commercially available in Turkish market by the time of the present invention.

### SUMMARY OF THE INVENTION

The object of this invention is to develop a pharmaceutical composition comprising otilonium bromide in combination with simethicone (in powder or in liquid form) wherein the dissolution properties of otilonium bromide are not hindered by the presence of simethicone used as the secondary active ingredient. Another object of this invention is to develop a pharmaceutical composition comprising otilonium bromide in combination with simethicone which will have advantageous attributes to overcome difficulties (e.g. compressibility) commonly encountered when simethicone is incorporated into solid pharmaceutical formulations.

Simethicone is known to cause difficulty in preparing free flowing, compressible solid dosage forms when substantial quantities of simethicone (either in solid or liquid form) is incorporated in the formulation. The difficulty can be a lack of sufficient cohesion in the compact for compression, particularly for direct compression tableting, such that the tablet will withstand the rigors of further processing, i.e., film-coating, printing, packaging and the like. Further, it can be difficult to assure that simethicone is uniformly distributed throughout the solid formulation and expeditiously dispersed upon administration.

Furthermore, simethicone is expected to decrease the dissolution rate of otilonium bromide, which may consequently hinder the therapeutic effect of otilonium bromide. The decrease of dissolution rate is more obvious at higher pH conditions (e.g., pH above 6.0).

It is the object of this invention to overcome this impact by using simethicone, hence to obtain improved dissolution rate of otilonium bromide, which consequently yields rapid and improved absorption of otilonium bromide throughout the GI tract and eventually desired symptomatic relief of spasm and pain is achieved.

The present invention provides compositions which may be easily and inexpensively formulated into solid dosage forms of otilonium bromide and simethicone treat patients suffering from irritable bowel syndrome (IBS) and/or pain and spasm of the distal enteric tract and/or painful symptoms of excessive gas in the GI tract. While tablets have been specifically named as a suitable and possible dosage form, other dosage forms also may be prepared according to the present invention. These additional dosage forms include but are not limited to film-coated tablets, dispersible tablets, orally disintegrating tablets, powders, and granules.

Another object of the present invention, therefore, is to provide compositions and processes that permit substantial quantities of simethicone to be incorporated into solid tablet formulations comprising otilonium bromide to be manufactured by various compression and other manufacturing processes.

In the scope of the present invention, various formulations and manufacturing processes have been investigated to prepare pharmaceutical combinations of otilonium bromide and simethicone. The weight ratio of otilonium bromide to simethicone is from about 1:1 to 1:5. Simethicone used may be in powder or in liquid form. The dissolution properties of said pharmaceutical combinations have also been studied at various pH's simulating gastrointestinal (GI) tract conditions. Surprisingly, it has been found that more than 85% of said otilonium bromide is released in 15 minutes in various dissolution media (i.e., 0.1N HCl, pH 4.5, pH 6.8 buffer solutions at 37±0.5° C.), and the final blend prior to compression has a bulk density of at least 0.35 g/mL, which enables final blends to be compressed into tablets.

In the scope of the present invention, two different methods have been investigated during the granulation process.

According to the first method, the inventors have attempted to minimize the interaction between simethicone and otilonium bromide to overcome the compressibility disadvantage and improve the dissolution characteristics of the tablets.

In a preferred embodiment of the present invention, an oral pharmaceutical composition comprising otilonium bromide in combination with simethicone, wherein
a. the final blend prior to compression has a bulk density of at least 0.35 g/mL, and
b. at least 85% of otilonium bromide is released in 15 minutes in 0.1 N HCl, pH 4.5, pH 6.8 buffer solutions at 37±0.5° C. using USP type II (paddle) apparatus rotating at 50 rpm.

In another preferred embodiment of the present invention, an oral pharmaceutical composition comprising otilonium bromide in combination with simethicone is formulated, wherein
a. otilonium bromide and simethicone are granulated together in the weight ratio of otilonium bromide to simethicone is from about 1:1 to 1:5, and
b. the final blend prior to compression has a bulk density of at least 0.35 g/mL, and
c. at least 85% of otilonium bromide is released in 15 minutes in 0.1 N HCl, pH 4.5, pH 6.8 buffer solutions simulating gastrointestinal (GI) tract conditions at 37±0.5° C. using USP type II (paddle) apparatus rotating at 50 rpm.

In another preferred embodiment of the present invention, an oral pharmaceutical composition comprising otilonium bromide in combination with simethicone is formulated, wherein
a. otilonium bromide and simethicone are granulated together in the weight ratio of otilonium bromide to simethicone is from about 1:1 to 1:5, and
b. the ratio of simethicone to mannitol or xylitol is about from 1:5 to 1:20, and
c. the final blend prior to compression has a bulk density of at least 0.35 g/mL, and
d. at least 85% of otilonium bromide is released in 15 minutes in 0.1 N HCl, pH 4.5, pH 6.8 buffer solutions simulating the gastrointestinal (GI) tract conditions at 37±0.5° C. using USP type II (paddle) apparatus rotating at 50 rpm.

In another preferred embodiment of the present invention, an oral pharmaceutical composition comprising otilonium bromide in combination with simethicone is formulated, wherein
a. otilonium bromide and simethicone are prepared as separate granules to prevent direct contact of simethicone with otilonium bromide, and
b. the final blend prior to compression has a bulk density of at least 0.35 g/mL, preferably at least 0.40 g/mL, and
c. at least 85% of otilonium bromide is released in 15 minutes in 0.1 N HCl, pH 4.5, pH 6.8 buffer solutions simulating the gastrointestinal (GI) tract conditions at 37±0.5° C. using USP type II (paddle) apparatus rotating at 50 rpm.

By means of the detailed description below, pharmaceutical combinations to be used via oral route may be developed in order to reach all the objects of the invention.

### DETAILED DESCRIPTION OF THE INVENTION

In the scope of the present invention various formulations comprising otilonium bromide and simethicone have been prepared using pharmaceutically acceptable excipients.

Suitable pharmaceutically acceptable excipients used according to this present invention include disintegrants, binders, lubricants, glidants, diluents/fillers, wetting agents, sweetening agents, flavoring agents and film-coating agents.

In one embodiment of the invention, the pharmaceutical composition comprises at least one disintegrant, selected from the group consisting of carboxymethylcellulose sodium, croscarmellose sodium, crospovidone, low substituted hydroxypropyl cellulose, magnesium aluminum silicate, methylcellulose, microcrystalline cellulose, sodium alginate, sodium starch glycolate, silicified microcrystalline cellulose, starch, pregelatinized starch, carboxymethylcellulose calcium and any combination thereof.

In one embodiment of the invention, the pharmaceutical composition comprises at least one binder, selected from the group consisting of carbomer, carboxymethylcellulose sodium, dextrin, dextrose, maltodextrin, gelatin, hydroxyethyl cellulose, hydroxyethylmethyl cellulose, hydroxypropyl cellulose, low substituted hydroxypropyl cellulose, ethylcellulose, methyl cellulose, hypromellose, magnesium aluminium silicate, methylcellulose, povidone, sodium alginate, starch, pregelatinised starch, liquid glucose, sucrose, tragacanth (gum benjamin), zein, acacia (gum arabic), alginic acid, guar gum, copovidone and any combination thereof.

In one embodiment of the invention, the pharmaceutical composition comprises at least one lubricant, selected from the group consisting of calcium stearate, glycerine monostearate, magnesium stearate, stearic acid, glyceryl palmitostearate, glyceryl behenate, hydrogenated castor oil, hydrogenated vegetable oil, sodium lauryl sulphate, magnesium lauryl sulphate, sodium stearyl fumarate, poloxamer, polyethylene glycol, sucrose ester of fatty acids, talc and any combination thereof.

In one embodiment of the invention, the pharmaceutical composition comprises at least one glidant, selected from the group consisting of tribasic calcium phosphate, calcium silicate, cellulose, colloidal silicon dioxide, magnesium silicate, magnesium tri silicate, starch, talc and any combination thereof.

In one embodiment of the invention, the pharmaceutical composition comprises at least one diluent/filler, selected from the group consisting of calcium carbonate, dibasic calcium phosphate anhydrous, calcium phosphate dihydrate, tribasic calcium phosphate, calcium sulphate, cellulose powdered, microcrystalline cellulose, silicified microcrystalline cellulose, cellulose acetate, colloidal silicon dioxide, dextrin, dextrose, lactose, magnesium carbonate, maltodextrin, mannitol, xylitol, polydextrose, starch, pregelatinized starch, calcium phosphate and any combination thereof. Preferred diluent/filler is mannitol.

In one embodiment of the invention, the pharmaceutical composition comprises at least one wetting agent, selected from the group consisting of gelatin, cetostearyl alcohol, polyoxyethylene castor oil derivatives, polyethylene glycols, sodium lauryl sulphate, poloxamer, carboxymethyl cellulose calcium, carboxymethyl cellulose sodium, methylcellulose, hydroxyethyl cellulose, hydroxylpropyl cellulose, hydroxypropylmethyl cellulose phthalate, magnesium aluminum silicate, triethanolamine, polyvinyl alcohol and any combination thereof.

In one embodiment of the invention, the pharmaceutical composition comprises otilonium bromide in combination with simethicone which is suitable for oral administration, such as tablet, film-coated tablet, dispersible tablet, orally disintegrating tablet, powder, granules or a combination thereof. Preferred dosage form is tablet.

The tablets may also contain sweetening agents such as aspartame, acesulfame potassium, saccharin sodium, cyclamates, sucralose, corn syrup, sorbitol (solution), maltitol (syrup), oligosaccharide, isomaltooligosaccharide, fructose, lactose, glucose, lycasin, xylitol, lactitol, erythritol, mannitol, isomaltose, polydextrose, dextrin or mixtures thereof.

The tablets may further contain natural and/or semi-synthetic or synthetic flavoring agents such as citrus, lemon, lime, orange, tangerine, grape fruit, peppermint, spearmint, apple, pear, plum, peach, apricot, strawberry, raspberry, cherry, banana, vanilla, watermelon, honey, bubble gum flavor or mixtures thereof.

Said pharmaceutical composition is compressed into tablets after direct compression or dry granulation or wet granulation. Water, ethanol, isopropyl alcohol and mixtures thereof can be used as solvents during wet granulation.

The tablets may be film-coated in a conventional manner. Suitable coatings include but not limited to hydroxypropylmethyl cellulose, ethyl cellulose, cellulose acetate, polyvinyl alcohol, acrylic and/or methacrylic co-polymers, resins, one or more plastifying agents (e.g., polyethylene glycol, propylene glycol, glycerin, triethyl citrate, diethyl phthalate, and mixtures thereof).

In a preferred embodiment of the invention, the pharmaceutical composition comprises otilonium bromide and simethicone in weight ratio from about 1:1 to 1:5, preferably from about 1:1 to 1:4, more preferably from about 1:1 to 1:3, most preferably about 1:2.

In one embodiment of the invention, mannitol and/or xylitol is used as the diluent/filler. The weight ratio of simethicone to mannitol and/or xylitol is about from 1:5 to 1:20, preferably from 1:5 to 1:10, more preferably from 1:5 to 1:8, most preferably is about 1:7. Other diluents/fillers may also be used.

Having described the invention with reference to certain embodiments, other embodiments will become apparent to the person skilled in the art from consideration of the specification. Certain specific aspects and embodiments of the invention will be further described in the following examples, which are provided solely for purposes of illustration and are not intended to limit the scope of the invention in any manner.

### Example 1

| | | | Unit Formula | |
|---|---|---|---|---|
| Formula I | | | (mg) | % |
| Wet | Phase I | Otilonium Bromide | 40.0 | 4.4 |
| Granulation | | Lactose Monohydrate | 797.0 | 88.6 |
| | | (Lactose 200 Mesh) | | |
| | | Sodium Starch | 18.0 | 2.0 |
| | | Glycolate (Primojel) | | |
| | Phase II | Corn Starch | 27.0 | 3.0 |

| | | | |
|---|---|---|---|
| Phase III | Magnesium Stearate | 18.0 | 2.0 |

| | | |
|---|---|---|
| Total Core Tablet Weight | 900.0 | 100 |

### Brief Manufacturing Process (Example 1):

Step-1: Ingredients in Phase I are mixed to form a homogeneous blend.

Step-2: The mixture in Step-1 is granulated with binder solution (Phase II). Obtained wet granules are dried and sifted.

Step-3: Lubricant (Phase III) is added to the granules from Step-2 and mixed.

Step-4: The granule blend from Step-3 is compressed into tablets.

### Example 2

| | | | Unit Formula | |
|---|---|---|---|---|
| Formula II | | | (mg) | % |
| Wet | Phase I | Otilonium Bromide | 40.0 | 4.4 |
| Granulation | | Simethicone (powder) | 80.0 | 8.9 |
| | | Lactose Monohydrate | 717.0 | 79.7 |
| | | (Lactose 200 Mesh) | | |
| | | Sodium Starch | 18.0 | 2.0 |
| | | Glycolate (Primojel) | | |
| | Phase II | Corn Starch | 27.0 | 3.0 |

| | | | |
|---|---|---|---|
| Phase III | Magnesium Stearate | 18.0 | 2.0 |

| | | |
|---|---|---|
| Total Core Tablet Weight | 900.0 | 100 |

### Manufacturing Process is Similar to as Described in Example 1.

The dissolution characteristics of tablets obtained from Example 1 and Example 2 are studied in various dissolution media simulating the gastrointestinal (GI) tract conditions (i.e., 0.1N HCl, pH 4.5, pH 6.8 buffer solutions at 37±0.5° C.), using the method described in Table 1. The mean values of otilonium bromide released in 15 minutes from Formula I and Formula II are compared in Table 2.

TABLE 1
Dissolution Method

| | |
|---|---|
| Equipment: | UV Spectrophotometer (wavelength: 260 nm) |
| Apparatus: | USP type II (paddle) |
| Rotation speed: | 50 rpm |
| Buffers: | Aqueous solutions of 0.1N hydrochloric acid/acetate |
| | buffer/phosphate buffer |
| Buffer volume: | 900 mL |
| Temperature: | 37° C. ± 0.5° C. |

TABLE 2
Released otilonium bromide in 15 minutes

| | Otilonium bromide 40 mg | Otilonium bromide 40 mg + |
|---|---|---|
| | Tablets | Simethicone 80 mg Tablets |
| | (Example 1) | (Example 2) |
| pH 1.2 | 91% | 81% |
| pH 4.5 | 90% | 60% |
| pH 6.8 | 86% | 43% |

It has been found that while more than 85% of said otilonium bromide is released in 15 minutes from the tablets comprising 40 mg of otilonium bromide as the mere active substance, tablets comprising 40 mg of otilonium bromide admixed with 80 mg of simethicone (powder) release significantly less amount of said otilonium bromide in 15 minutes under same pH conditions. The decrease of dissolution rate is more obvious at higher pH conditions.

### Example 3

| | | | Unit Formula | |
|---|---|---|---|---|
| Formula III | | | (mg) | % |
| Wet | Phase I | Microcrystalline | 445 | 49.4 |
| Granulation | | Cellulose | | |
| | | (Avicel PH102) | | |
| | | Crospovidone | 45 | 5.0 |
| | | (Polyplasdone XL-10) | | |
| | | Colloidal Silicon | 20 | 2.2 |
| | | Dioxide (Aerosil 300) | | |
| | Phase II | Simethicone (liquid) | 80 | 8.9 |
| | | Copovidone | 27 | 3.0 |
| | | (Kollidon VA 64) | | |

| | | | |
|---|---|---|---|
| Phase III | Otilonium Bromide | 40 | 4.4 |
| | Lactose, Spray Dried | 213 | 23.7 |
| | Copovidone | 18 | 2.0 |
| | (Kollidon VA 64) | | |
| | Colloidal Silicon | 7 | 0.8 |
| | Dioxide (Aerosil 200) | | |
| | Magnesium Stearat | 5 | 0.6 |

| | | |
|---|---|---|
| Total Core Tablet Weight | 900.0 | 100.0 |

### Brief Manufacturing Process (Example 3):

Step-1: Microcrystalline cellulose, crospovidone, and colloidal silicon dioxide are mixed to form a homogeneous blend.

Step-2: The mixture in Step-1 is granulated with simethicone (liquid)

Step-3: The mixture in Step-2 is further granulated with binder solution comprising copovidone. Obtained wet granules are dried and sifted.

Step-4: Otilonium bromide, lactose spray dried, copovidone, and colloidal silicon dioxide are added to the granules from Step-3 and mixed.

Step-5: Magnesium stearate is added to the granules from Step-4 and mixed.

However, the final blend as prepared in Example 3 is not compressible into tablets. The bulk density of the final blend prior to compression is measured to be 0.32 g/mL.

### Example 4

| | | | Unit Formula | |
|---|---|---|---|---|
| Formula IV | | | (mg) | % |
| Wet | Phase I | Otilonium Bromide | 40.0 | 4.4 |
| Granulation | | Mannitol | 50.0 | 5.6 |
| | | Microcrystalline | 625.0 | 69.5 |
| | | Cellulose | | |
| | | (Avicel PH101) | | |
| | | Crospovidone | 30.0 | 3.3 |
| | | (Polyplasdone XL-10) | | |
| | | Colloidal Silicon | 20.0 | 2.2 |
| | | Dioxide (Aerosil 200) | | |
| | | Colloidal Silicon | 20.0 | 2.2 |
| | | Dioxide (Aerosil 300) | | |
| | Phase II | Simethicone (liquid) | 80.0 | 8.9 |
| | | Povidone (PVP K30) | 30.0 | 3.3 |

| | | | |
|---|---|---|---|
| Phase III | Magnesium Stearate | 5.0 | 0.6 |

| | | |
|---|---|---|
| Total Core Tablet Weight | 900.0 | 100.0 |

### Brief Manufacturing Process (Example 4):

Step-1: Ingredients in Phase I are mixed to form a homogeneous blend.

Step-2: The mixture in Step-1 is granulated with binder solution comprising povidone and liquid simethicone (Phase II). Obtained wet granules are dried and sifted.

Step-3: Magnesium stearate (Phase III) is added to the granules from Step-2 and mixed.

However, the final blend as prepared in Formula IV (Example 4) is not compressible into tablets. The bulk density of the final blend prior to compression as prepared in Example 4 is measured to be 0.32 g/mL and the weight ratio of simethicone to mannitol is about 1:0.6. The effect of amount of mannitol as a diluent/filler is investigated in Examples 5 and 6.

### Example 5

| | | | Unit Formula | |
|---|---|---|---|---|
| Formula V | | | (mg) | % |
| Wet | Phase I | Otilonium Bromide | 40.0 | 4.0 |
| Granulation | | Mannitol | 200.0 | 20.0 |
| | | Lactose Monohydrate | 620.0 | 62.0 |
| | | (Lactose 200 Mesh) | | |
| | | Microcrystalline | 30.0 | 3.0 |
| | | Cellulose | | |
| | | (Avicel PH101) | | |
| | Phase II | Simethicone (liquid) | 80.0 | 8.0 |
| | | Povidone (PVP K30) | 25.0 | 2.5 |

| | | | |
|---|---|---|---|
| Phase III | Magnesium Stearate | 5.0 | 0.5 |

| | | |
|---|---|---|
| Total Core Tablet Weight | 1000.0 | 100 |

Manufacturing process is similar to as described in Example 4.

The bulk density of the final blend prior to compression as prepared in Formula V (Example 5) is measured to be 0.55 g/mL and the weight ratio of simethicone to mannitol is 1:2.5. The dissolution characteristics of tablets obtained from Example 5 are studied in solutions of pH 1.2, 4.5 and 6.8 using the method described in Table 1. The mean values of otilonium bromide released in 15 minutes are given in Table 3.

TABLE 3
Released otilonium bromide in 15 minutes

| | Otilonium bromide 40 mg + |
|---|---|
| | Simethicone 80 mg Tablets |
| | (Example 5) |
| pH 1.2 | 88% |
| pH 4.5 | 86% |
| pH 6.8 | 61% |

Surprisingly, the amount of otilonium bromide released in 15 minutes increased to the desired level (>85%) at pH 1.2 and 4.5, but not in pH 6.8. This suggests that the amount of mannitol used in the formula with respect to simethicone needs to be increased even more, hence, the dissolution characteristics of otilonium bromide will be further improved to become more than 85% in 15 minutes not only pH 1.2 and 4.5, but also at pH 6.8. This is demonstrated in Example 6.

### Example 6

| | | | Unit Formula | |
|---|---|---|---|---|
| Formula VI | | | (mg) | % |
| Wet | Phase I | Otilonium Bromide | 40.0 | 4.4 |
| Granulation | | Mannitol | 575.0 | 63.9 |
| | | Lactose Monohydrate | 145.0 | 16.1 |
| | | (Lactose 200 Mesh) | | |
| | | Microcrystalline | 30.0 | 3.3 |
| | | Cellulose | | |
| | | (Avicel PH101) | | |
| | Phase II | Simethicone (liquid) | 80.0 | 8.9 |
| | | Povidone (PVP K30) | 25.0 | 2.8 |

| | | | |
|---|---|---|---|
| Phase III | Magnesium Stearate | 5.0 | 0.6 |

| | | |
|---|---|---|
| Total Core Tablet Weight | 900.0 | 100 |

Manufacturing process is similar to as described in Example 4.

The bulk density of the final blend prior to compression as prepared in Formula VI (Example 6) is measured to be 0.52 g/mL and the weight ratio of simethicone to mannitol is 1:7.2. The dissolution characteristics of tablets obtained from Example 6 are studied in solutions of pH 1.2, 4.5 and 6.8 using the method described in Table 1. The mean values of otilonium bromide released in 15 minutes are given in Table 4. The amount of otilonium bromide released in 15 minutes reached the desired level (>85%) at all three pH conditions.

TABLE 4
Released otilonium bromide in 15 minutes

| | Otilonium bromide 40 mg + |
|---|---|
| | Simethicone 80 mg Tablets |
| | (Example 6) |
| pH 1.2 | 92% |
| pH 4.5 | 89% |
| pH 6.8 | 87% |

### Example 7

Alternatively, xylitol can be used in the formulation instead of mannitol. This is demonstrated in Example 7.

| | | | Unit Formula | |
|---|---|---|---|---|
| Formula VII | | | (mg) | % |
| Wet | Phase I | Otilonium Bromide | 40.0 | 4.4 |
| Granulation | | Xylitol | 575.0 | 63.9 |
| | | Lactose Monohydrate | 145.0 | 16.1 |
| | | (Lactose 200 Mesh) | | |
| | | Microcrystalline | 30.0 | 3.3 |
| | | Cellulose | | |
| | | (Avicel PH101) | | |
| | Phase II | Simethicone (liquid) | 80.0 | 8.9 |
| | | Povidone (PVP K30) | 25.0 | 2.8 |

| | | | |
|---|---|---|---|
| Phase III | Magnesium Stearate | 5.0 | 0.6 |

| | | |
|---|---|---|
| Total Core Tablet Weight | 900.0 | 100 |

Manufacturing process is similar to as described in Example 4.

The dissolution characteristics of tablets obtained from Example 7 are studied in solutions of pH 1.2, 4.5 and 6.8 using the method described in Table 1. The mean values of otilonium bromide released in 15 minutes are given in Table 5. The amount of otilonium bromide released in 15 minutes reached the desired level (>85%) at all three pH conditions.

TABLE 5
Released otilonium bromide in 15 minutes

| | Otilonium bromide 40 mg + |
|---|---|
| | Simethicone 80 mg Tablets |
| | (Example 7) |
| pH 1.2 | 90% |
| pH 4.5 | 88% |
| pH 6.8 | 86% |

### Example 8

| | | | Unit Formula | |
|---|---|---|---|---|
| Formula VIII | | | (mg) | % |
| Wet | Phase I | Microcrystalline | 575 | 63.9 |
| Granulation | | Cellulose | | |
| I | | (Avicel PH101) | | |
| | | Crospovidone | 30 | 3.4 |
| | | (Polyplasdone XL-10) | | |
| | | Colloidal Silicon | 20 | 2.2 |
| | | Dioxide (Aerosil 300) | | |
| | Phase II | Simethicone (liquid) | 80 | 8.9 |
| | | Copovidone | 18 | 2.0 |
| | | (Kollidon VA 64) | | |
| Wet | Phase III | Otilonium Bromide | 40 | 4.4 |
| Granulation | | Lactose Monohydrate | 100 | 11.1 |
| II | | (Lactose 200 Mesh) | | |
| | | Copovidone | 12 | 1.3 |
| | | (Kollidon VA 64) | | |

| | | | |
|---|---|---|---|
| Phase IV | Colloidal Silicon | 20 | 2.2 |
| | Dioxide (Aerosil 200) | | |
| | Magnesium Stearate | 5 | 0.6 |

| | | |
|---|---|---|
| Total Core Tablet Weight | 900 | 100 |

### Brief Manufacturing Process (Example 8):

Step-1: Ingredients in Phase I are mixed to form a homogeneous blend.

Step-2: The mixture in Step-1 is granulated with binder solution comprising copovidone and liquid simethicone (Phase II). Obtained wet granules are dried and sifted.

Step-3: Ingredients in Phase III are mixed and granulated with water. The wet granules obtained are dried and sieved.

Step-4: The mixture from Step-2 and Step-3 are mixed.

Step-5: Ingredients in Phase IV are added to the mixture in Step-4 and mixed further to form a homogeneous blend.

However, the final blend as prepared in Example 8 is not compressible into tablets. The bulk density of the final blend prior to compression is measured to be 0.30 g/mL.

### Example 9

| | | | Unit Formula | |
|---|---|---|---|---|
| Formula IX | | | (mg) | % |
| Wet | Phase I | Microcrystalline | 445.0 | 49.4 |
| Granulation | | Cellulose | | |
| I | | (Avicel PH102) | | |
| | | Crospovidone | 45.0 | 5.0 |
| | | (Polyplasdone XL-10) | | |
| | | Colloidal Silicon | 20.0 | 2.2 |
| | | Dioxide (Aerosil 300) | | |
| | | Simethicone (powder) | 80.0 | 8.9 |
| | Phase II | Copovidone | 27.0 | 3.0 |
| | | (Kollidon VA 64) | | |
| Wet | Phase III | Otilonium Bromide | 40.0 | 4.4 |
| Granulation | | Lactose Monohydrate | 213.0 | 23.7 |
| II | | (Lactose 200 Mesh) | | |
| | | Copovidone | 18.0 | 2.0 |
| | | (Kollidon VA 64) | | |

| | | | |
|---|---|---|---|
| Phase IV | Colloidal Silicon | 7.0 | 0.8 |
| | Dioxide (Aerosil 200) | | |
| | Magnesium Stearate | 5.0 | 0.6 |

| | | |
|---|---|---|
| Total Core Tablet Weight | 900.0 | 100 |

### Brief Manufacturing Process (Example 9):

Step-1: Ingredients in Phase I are mixed to form a homogeneous blend.

Step-2: The mixture in Step-1 is granulated with binder solution comprising copovidone (Phase II). Obtained wet granules are dried and sifted.

Step-3: Ingredients in Phase III are mixed and granulated with water. The wet granules obtained are dried and sieved.

Step-4: The mixture from Step-2 and Step-3 are mixed.

Step-5: Ingredients in Phase IV are added to the mixture in Step-4 and mixed further to form a homogeneous blend.

Step-6: The granule blend from Step-5 is compressed into tablets.

The bulk density of the final blend prior to compression as prepared in Formula IX (Example 9) is measured to be 0.41 g/mL. The dissolution characteristics of tablets obtained from Example 9 are studied in solutions of pH 1.2, 4.5 and 6.8 using the method described in Table 1. The mean values of otilonium bromide released in 15 minutes are given in Table 6. The amount of otilonium bromide released in 15 minutes reached the desired level (>85%) at all three pH conditions.

TABLE 6
Released otilonium bromide in 15 minutes

| | Otilonium bromide 40 mg + |
|---|---|
| | Simethicone 80 mg Tablets |
| | (Example 9) |
| pH 1.2 | 95% |
| pH 4.5 | 87% |
| pH 6.8 | 86% |

Alternatively, liquid simethicone can be used in the formulation instead of powder simethicone. Tl is demonstrated in Example 10.

### Example 10

| | | | Unit Formula | |
|---|---|---|---|---|
| Formula X | | | (mg) | % |
| Wet | Phase I | Microcrystalline | 445.0 | 49.4 |
| Granulation | | Cellulose | | |
| I | | (Avicel PH102) | | |
| | | Crospovidone | 45.0 | 5.0 |
| | | (Polyplasdone XL-10) | | |
| | | Colloidal Silicon | 20.0 | 2.2 |
| | | Dioxide (Aerosil 300) | | |
| | Phase II | Simethicone (liquid) | 80.0 | 8.9 |
| | | Copovidone | 27.0 | 3.0 |
| | | (Kollidon VA 64) | | |
| Wet | Phase III | Otilonium Bromide | 40.0 | 4.4 |
| Granulation | | Lactose Monohydrate | 213.0 | 23.7 |
| II | | (Lactose 200 Mesh) | | |
| | | Copovidone | 18.0 | 2.0 |
| | | (Kollidon VA 64) | | |

| | | | |
|---|---|---|---|
| Phase IV | Colloidal Silicon | 7.0 | 0.8 |
| | Dioxide (Aerosil 200) | | |
| | Magnesium Stearate | 5.0 | 0.6 |

| | | |
|---|---|---|
| Total Core Tablet Weight | 900.0 | 100 |

### Brief Manufacturing Process (Example 10):

Step-1: Microcrystalline cellulose, crospovidone, and colloidal silicon dioxide are mixed to form a homogeneous blend.

Step-2: Simethicone (liquid) is added to the mixture from Step-1 and mixed.

Step-3: The mixture in Step-2 is granulated with binder solution comprising copovidone. Obtained wet granules are dried and sifted.

Step-4: Otilonium bromide, lactose monohydrate and copovidone are mixed and granulated with water. The wet granules obtained are dried and sieved.

Step-5: The mixture from Step-3 and Step-4 are mixed.

Step-6: Colloidal silicon dioxide and magnesium Stearate are added to the mixture in Step-5 and mixed further to form a homogeneous blend.

Step-7: The granule blend from Step-6 is compressed into tablets.

The bulk density of the final blend prior to compression as prepared in Formula X (Example 10) is measured to be 0.42 g/mL. The dissolution characteristics of tablets obtained from Example 10 are studied in solutions of pH 1.2, 4.5 and 6.8 using the method described in Table 1. The mean values of otilonium bromide released in 15 minutes are given in Table 7. The amount of otilonium bromide released in 15 minutes reached the desired level (>85%) at all three pH conditions.

TABLE 7
Released otilonium bromide in 15 minutes

| | Otilonium bromide 40 mg + |
|---|---|
| | Simethicone 80 mg Tablets |
| | (Example 10) |
| pH 1.2 | 100% |
| pH 4.5 | 99% |
| pH 6.8 | 94% |

The inventors have concluded that that when the interaction between simethicone and otilonium bromide is minimized/prevented, the compressibility disadvantage is conquered and the dissolution characteristics of the tablets are improved.

According to the first method of the invention, when the ratio of otilonium bromide to simethicone is from about 1:1 to 1:5, and the ratio of simethicone to mannitol is about from 1:5 to 1:20, it enables more than 85% of said otilonium bromide to be released in 15 minutes in 0.1 N HCl, pH 4.5, pH 6.8 buffer solutions at 37±0.5° C. using USP type II (paddle) apparatus rotating at 50 rpm, and the final blend prior to compression has a bulk density of at least 0.35 g/mL.

According to the second method of the invention when otilonium bromide and simethicone are prepared as separate granules to prevent direct contact of simethicone with otilonium bromide, it enables more than 85% of otilonium bromide to be released in 15 minutes in 0.1 N HCl, pH 4.5, pH 6.8 buffer solutions at 37±0.5° C. using USP type II (paddle) apparatus rotating at 50 rpm, and the final blend prior to compression has a bulk density of at least 0.40 g/mL.

While the invention has been described with respect to the above specific embodiments, it should be recognized that various modifications and changes may be made to the invention by those skilled in the art which also fall within the scope of the invention as defined by the appended claims.

1. An oral pharmaceutical composition comprising otilonium bromide in combination with simethicone, wherein
a final blend prior to compression has a bulk density of at least 0.35 g/mL; and
at least 85% of the otilonium bromide is released in 15 minutes in each of 0.1 N HCl, pH 4.5 and pH 6.8 buffer solutions at 37±0.5° C. using USP type II (paddle) apparatus rotating at 50 rpm.

2. The oral pharmaceutical composition according to claim 1, wherein the final blend prior to compression has a bulk density of at least 0.40 g/mL.

3. The oral pharmaceutical composition according to claim 1, wherein the oral pharmaceutical composition further comprises mannitol and a weight ratio of the simethicone to the mannitol is from 1:5 to 1:20.

4. The oral pharmaceutical composition according to claim 3, wherein the weight ratio of the simethicone to the mannitol is from 1:5 to 1:10.

5. The oral pharmaceutical composition according to claim 1, wherein the oral pharmaceutical composition further comprises xylitol and a weight ratio of the simethicone to the xylitol is from 1:5 to 1:20.

6. The oral pharmaceutical composition according to claim 5, wherein the weight ratio of the simethicone to the xylitol is from 1:5 to 1:10.

7. The oral pharmaceutical composition according to claim 1, wherein the simethicone is in liquid form.

8. The oral pharmaceutical composition according to claim 1, wherein the oral pharmaceutical composition comprises the otilonium bromide and the simethicone in separate granules.

9. The oral pharmaceutical composition according to claim 1, wherein the oral pharmaceutical composition is a tablet, a film-coated tablet, a dispersible tablet, an orally disintegrating tablet, a powder, a granule or a combination thereof.

10. The oral pharmaceutical composition according to claim 9, wherein oral pharmaceutical composition is the tablet.

11. (canceled)

12. (canceled)

13. The oral pharmaceutical composition according to claim 1, wherein a weight ratio of the otilonium bromide to the simethicone is from 1:1 to 1:5.

14. The oral pharmaceutical composition according to claim 1, wherein the oral pharmaceutical composition comprises the otilonium bromide in an amount of 40 mg and the simethicone in an amount of 80 mg per oral dosage unit.

15. The oral pharmaceutical composition according to claim 1, wherein the oral pharmaceutical composition is used for a treatment or alleviating a symptoms of irritable bowel syndrome (IBS) and pain/spasm in a gastrointestinal tract.

16. The oral pharmaceutical composition according to claim 4, wherein the weight ratio of the simethicone to the mannitol is from 1:5 to 1:8.

17. The oral pharmaceutical composition according to claim 16, wherein the weight ratio of the simethicone to the mannitol is 1:7.

18. The oral pharmaceutical composition according to claim 6, wherein the weight ratio of the simethicone to the xylitol is from 1:5 to 1:8.

19. The oral pharmaceutical composition according to claim 18, wherein the weight ratio of the simethicone to the xylitol is 1:7.

20. The oral pharmaceutical composition according to claim 13, wherein a weight ratio of the otilonium bromide to the simethicone is from 1:1 to 1:4

21. The oral pharmaceutical composition according to claim 20, wherein the weight ratio of the otilonium bromide to the simethicone is more preferably from 1:1 to 1:3.

22. The oral pharmaceutical composition according to claim 21, wherein the weight ratio of the otilonium bromide to the simethicone is 1:2.
